SEQUENCE LISTING (12 sequences)

SEQ ID NO: 1 - protein, 21 aa, synthetic construct
  REGION 1..21: note = Sequence is synthesized
WWFWIWFWWGPGRRKRRKRRR

SEQ ID NO: 2 - protein, 22 aa, synthetic construct
  REGION 1..22: note = Sequence is synthesized
WWFLSIWFLWGPGRRKRRKRRR

SEQ ID NO: 3 - protein, 21 aa, synthetic construct
  REGION 1..21: note = Sequence is synthesized
WLFWIWVWWGPGRRKFRKRAR

SEQ ID NO: 4 - protein, 21 aa, synthetic construct
  REGION 1..21: note = Sequence is synthesized
WLFWIWFWSGPGRRLKRKVRK

SEQ ID NO: 5 - protein, 21 aa, synthetic construct
  REGION 1..21: note = Sequence is synthesized
WWFWIWFWWGSGRRKRRKRRR

SEQ ID NO: 6 - protein, 24 aa, synthetic construct
ILVWWFWIWFWWASTRRLRRKRRR

SEQ ID NO: 7 - DNA, 38 bp, synthetic construct
  source 1..38: mol_type = other DNA
agcaagccatgggcatgactcgcgatttcaaacctgga

SEQ ID NO: 8 - DNA, 39 bp, synthetic construct
  source 1..39: mol_type = other DNA
agcaagaagcttctactgctcagtttccatttgttcctc

SEQ ID NO: 9 - DNA, 101 bp, synthetic construct
  source 1..101: mol_type = other DNA
agcaagccatgggctggtggttttggatttggttttggtggggcccgggccgccgcaaacgccgcaaacgccgccgcatgactcgcgatttcaaacctgga

SEQ ID NO: 10 - DNA, 100 bp, synthetic construct
  source 1..100: mol_type = other DNA
aagctgccatgggctggtggttttggatttggttttggtggggcccgggccgccgcaaacgccgcaaacgccgccgcatggacatcgccatccaccaccc

SEQ ID NO: 11 - DNA, 31 bp, synthetic construct
  source 1..31: mol_type = other DNA
aagctgccatggacatcgccatccaccaccc

SEQ ID NO: 12 - DNA, 36 bp, synthetic construct
  source 1..36: mol_type = other DNA
gagagacatatgctatttcttgggggctgcggtgac

(19) United States
(12) Patent Application Publication
(10) Pub. No.: US 2024/0092837 A1
(43) Pub. Date: Mar. 21, 2024

(54) Novel Peptide Carrier Compositions

(71) Applicant: The Regents of the University of Colorado, A Body Corporate, Denver, CO (US)

(72) Inventors: Uday Kompella, Centennial, CO (US); Arun Upadhyay, West Chester, PA (US)

(73) Assignee: The Regents of the University of Colorado, A Body Corporate, Denver, CO (US)

(21) Appl. No.: 18/359,793

(22) Filed: Jul. 26, 2023

Related U.S. Application Data
(63) Continuation of application No. PCT/US2022/013851, filed on Jan. 26, 2022.
(60) Provisional application No. 63/142,219, filed on Jan. 27, 2021.

(51) Int. Cl. C07K 14/00; A61K 47/64

(52) U.S. Cl. CPC C07K 14/001; A61K 47/64

(57) ABSTRACT

The present invention describes peptides, peptides carriers, peptide nanobodies, and peptide-drug covalent conjugates having efficient cell and tissue penetration. The peptides and associated configurations can be used in covalent attachments or as complexes or nanoparticles in conjunction with therapeutic agents to enhance their tissue, cellular, and intracellular delivery. Also, the peptides and associated configurations can enhance binding to negatively charged matrices in the body for improved localization or retention of therapeutic carriers and agents.

### CROSS-REFERENCE TO RELATED APPLICATIONS

This application is a continuation of and claims priority to co-pending Patent Cooperation Treaty International Application Serial No. PCT/US2022/013851, filed Jan. 26, 2022, designating the United States, which claims priority to U.S. Provisional Patent Application Ser. No. 63/142,219, filed Jan. 27, 2021.

### SEQUENCE LISTING

This application contains a Sequence Listing which has been submitted in XML file format with this application, the entire content of which is incorporated by reference herein in its entirety. The Sequence Listing XML file submitted with this application is entitled “6470-018.US_SEQ_Replacement.xml”, was created on Dec. 4, 2023, and is 13,867 bytes in size.

### BACKGROUND OF THE INVENTION

Delivery of therapeutic and diagnostic agents, and their carriers including membrane attachment, membrane entry, and passage across the membranes in order to access targeted cells, tissues, and organelles remains a very challenging task to realize the full potential of these agents. The plasma membrane acts as a natural gatekeeper for the transportation of molecules and carriers across its boundary; this in some cases protects the organisms from a number of environmental insults. This selective permeability of plasma and intracellular membranes is due to their structural design wherein a resistant lipid bilayer is impregnated with a variety of proteins and other biomolecules that form a relatively impenetrable barrier. While the primary lipid component is very hydrophobic in nature, the primary protein component imparts a hydrophilic nature to biological barriers. The gaps between the various components in a membrane are sufficiently small to exclude most solute from entering the cell or intracellular compartments. Thus, a molecule or carrier should possess unique attributes to interact with the membranes for effective agent delivery. This is an ongoing task with any new therapeutic, diagnostic, or carrier agent as these agents are advanced for biological use. Therefore, no simple rule encompasses all approaches to enable biological barrier crossing. Also, no single approach will result in enhanced delivery for all potential agents that might be introduced.

Peptide and protein drug molecules, collectively referred to herein as “proteins), are large molecules composed of amino acids and form complex two- and three-dimensional structures. As previously mentioned, proteins are typically hydrophilic on the surface. Due to their large size as well as their hydrophilic nature, biological barriers are difficult for proteins to enter and/or cross. While the presence of cell surface receptors that help in internalization and transport of selective molecules across membrane barrier provide a possible mechanism for the entry of proteins, this mechanism may be inefficient or not present for most practical purposes. Similar to proteins, nucleic acid agents are also large and hydrophilic, and have great difficulty in crossing the biological barriers. Therefore, the need exists to develop new methods to increase delivery of macromolecules, proteins and other agents.

Small molecule therapeutic agents and molecules are also delivered to a small extent or not at all across a variety of biological barriers. Bioavailability of the drug or diagnostic agent at the targeted site relative to what was initially dosed is extremely small, which leaves much room for improvement in transmembrane drug transport. This problem is amplified as the size of the molecule, if hydrophilicity increases, or if the solubility decreases. Additionally, delivery becomes even more limited when the agent or carrier has a short contact time with the sites of drug entry. For this reason, there is also a continuing need to enhance or increase delivery of small molecules across biological barriers by improving their solubility and/or attachment, so that entry into and passage across the biological membranes can be increased.

A variety of carriers can be used to deliver therapeutic, diagnostic, or other agents to biological systems. These carriers may not be delivered efficiently to biological membranes due to their size and surface properties. These carriers can include macromolecules that are linked to or associated with an agent, colloidal systems including nanoparticles that are associated with an agent, or carriers such as microsystems (e.g., microspheres and objects of other shapes) and macrosystems (e.g., implants). To anchor, allow the entry of, or to allow the passage of such carrier systems across biological barriers, requires the use of special adaptations.

Historically, various methods have been developed and tested to enhance the delivery of macromolecules by such as by altering the physicochemical properties to macromolecules, using different carriers, e.g., nanobodies or dendrimers, and enhanced cell transportation peptides. Among these, the use of small enhanced cell transportation peptides have been emerging as a very promising tools for enhanced intracellular cargo delivery. Since the discovery of the transactivator of transcription (TAT) protein from the Human immunodeficiency virus 1 (HIV-1), many other peptides with various characteristics exhibiting enhanced cell penetration properties have been identified. Most of the cell penetration peptides (CPPs) were derived from membrane interacting proteins, transmembrane proteins, and signal peptides of secretory proteins. These proteins are composed of a small stretch of amino acid residues having properties which enable them to cross through the plasma membrane, known as protein transduction domains (PTD). These PTDs have been shown to facilitate the crossing of biological membranes without the help of any receptors. The most common peptides in this category include the TAT peptide from HIV, the antennapedia homeodomain protein from drosophila, transportans, and VP22 from herpes simplex virus.

Besides these PTD based peptides, other peptides with cell enhanced transportation properties have also been designed and synthesized. Such peptides were designed with specific physicochemical properties so that they can interact strongly with the plasma membrane and at the same time can make their way through the hydrophobic boundary of cellular cytoplasm. These peptides were designed to have amphipathic, cationic hydrophilic, anionic hydrophilic, hydrophobic properties or a combination thereof. For instance, the amphipathic peptides were designed in such a way that hydrophobic and hydrophilic moieties are positioned at opposite faces of peptide secondary structure or they are arranged linearly in the primary structure.

Due to the compositional nature of cell peptides there are many limitations in terms of their delivery and toxicity in various cell types during cargo delivery. These peptides are known to cause toxic effects such as; rupturing of the membrane causing cytoplasmic leakage and cell death, host cell metabolic burden, and the activation of immunologic responses. Most of the previously described peptides for enhanced cell penetration are limited by their efficiency to deliver therapeutic cargos across tissue barriers and cellular toxicity.

In accordance with the present disclosure there is provided a novel peptide design comprising a positive, helical, and hydrophobic peptide (PHHP) having at least one amphipathic beta strand-turn-alpha helix (PTa) motif domain. For ease of reference, this novel peptide design will be referred to alternatively as the PHHP peptide(s) or PHHP variant peptide(s) or as P 1 to P 6 for each of six alternative variations of the PHHP peptides. In addition to their enhanced transportation and penetration characteristics, the inventive PHHP peptides also exhibit reduced cell and/or tissue toxicity without having homological similarities to existing natural molecules.

Some penetrating peptides can get entrapped in cellular compartments like the endosomes or even the lysosomes which can then lead to the degradation of cargo molecules, more specifically peptides, proteins, and nucleic acids. This limitation hinders the therapeutic potential of cargo molecules intended for cytoplasmic or even nuclear targets. In some cases, the cargo may not be released by cell enhanced penetrating peptide cargo vehicles which also reduce their therapeutic potential.

There are currently about sixty peptide and protein drugs that have been approved by the United States Food and Drug Administration (FDA). Since cell and other membranes represent key barriers for drug delivery, the majority of these FDA approved macromolecules act on the cell surface. The present invention can benefit these and other molecules whose delivery and/or activity is limited by cell and tissue barriers.

### SUMMARY OF THE INVENTION

An objective of the present invention is to provide peptide sequences that facilitate biomolecule or membrane attachment, membrane entry, and membrane passage of a variety of agents including macromolecules, small molecules, and other carrier systems.

It is a further objective of the present invention to provide peptides capable of being assembled into a variety of structures or carriers in order to enable the delivery of any associated agents to the desired tissue locations.

It is a still further objective of the present invention to provide a gene construct wherein a model protein coding sequence is joined within a representative peptide coding sequence and expressed in a model host cell system which may then be purified.

It is yet still another objective of the present invention to assess the resultant expressed protein for its ability to enhance delivery in mammalian cell lines, e.g., ARPE-19, A549, or hCET, and tissue models.

It is still another objective of the present invention to demonstrate enhanced delivery for two proteins, i.e., human alpha crystallin B and human lens epithelium derived growth factor variants (LEDGF₍₁₋₃₂₆₎).

It is another objective of the present invention to provide peptide sequences that are also configurable to form nano- and micro-structures for small molecule delivery.

It is a further objective of the present invention is to provide peptide sequences capable of forming micelles that solubilize small molecule solutes and enhance delivery of the small molecule solute, and nucleic acids.

It is yet a still further objective of the present invention to attach the inventive peptide sequences to carrier systems to enhance carrier delivery. As an example, polymeric nanoparticles were loaded with a model solute then associated with the inventive peptide sequences and the transmembrane delivery of the polymeric nanoparticle was evaluated using cultured cells.

Finally, it is another objective of the present invention to employ the inventive peptides as anchor materials to negatively charged matrices, such as the vitreous humor of the eye or other tissue surfaces, in order to achieve improved localization and prolonged retention of the therapeutic agent complexed with the peptide.

These and other objects, features, and advantages of the present invention will become more apparent to those skilled in the art from the following more detailed description of the invention with reference to the accompanying drawings and experimental data.

### BRIEF DESCRIPTION OF THE FIGURES

FIG. 1A sets forth Peptide sequences, physiochemical properties data, and Kyte-Doolittle Hydropathy Plots of PHHP peptide variants (P 1 to P 6) along with the TAT control peptide.

FIG. 1B are PEP-FOLD models with the peptide sequence data generated by the data shown in FIG. 1A.

FIG. 2 are graphs illustrating the effect of buffer pH, salt, and surfactant on the size of PHHP peptides nanoassemblies and TAT control peptide; Panel A is without salt and the Tween-80 surfactant; Panel B is with salt, 0.5 M NaCl and 0.1% Tween-80 surfactant.

FIG. 3 is a graph of intracellular uptake of the PHHP peptide variants (P 1 to P 6) in ARPE-19 cells relative to the TAT control peptide measured using a fluorometric method,

FIG. 4. Is a graph of intracellular uptake of the PHHP peptide variants (P 1 to P 6) in AR-PE-19 cells relative to TAT using fluorescence microscopy.

FIG. 5 are color photographs illustrating uptake of PHHP variant peptides (P 1 to P 6) along with the control peptide TAT in ARPE-19 cells. The uptake duration was 1 hour at a 10 μM peptide concentration and incubation volume of 200 μl per well in a 96 well culture plate.

FIG. 6 are graphs illustrating quantitation of the in vitro cell uptake study of Alexa Flour® 488 labeled CRYAB, PHIP (P 1)-CRYAB, LEDGF₍₁₋₃₂₆₎, and PHHP (P 1)-LEDGF₍₁₋₃₂₆₎ in three different cell lines; ARPE-19 (human retinal pigment epithelium) cells, A549 (adenocarcinomic human alveolar basal epithelial) cells, and hCET (telomerase-immortalized human corneal epithelial) cells. Panel A illustrates uptake of CRYAB and PHHP (P 1)-CRYAB in ARPE-19 cells, A549 cells, and hCET cells at different time points. Panel B illustrates uptake of LEDGF₍₁₋₃₂₆₎ and PHHP (P 1)-LEDGF₍₁₋₃₂₆₎ in ARPE-19 cells, A549 cells, and hCET cells at different time points.

FIG. 7A illustrates the results of an in vitro cell uptake study of Alexa Flour® 488 labeled LEDGF₍₁₋₃₂₆₎, and PHHP (P 1)-LEDGF₍₁₋₃₂₆₎, in three different cell lines; ARPE-19 (human retinal pigment epithelium) cells (Section A), A549 (adenocarcinomic human alveolar basal epithelial) cells (Section B), and hCET (telomerase-immortalized human corneal epithelial) cells (Section C).

FIG. 7B illustrates results of an in vitro cell uptake study of Alexa Flour® 488 labeled B) CRYAB, and PHHP (P 1)-CRYAB in three different cell lines; ARPE-19 (human retinal pigment epithelium) cells (Section A), A549 (adenocarcinomic human alveolar basal epithelial) cells (Section B), and hCET (telomerase-immortalized human corneal epithelial) cells (Section C).

FIG. 8 are graphs of ex-vivo transcellular migration and uptake study of Alexa Fluor® 488 labeled CRYAB, PHHP (P 1)-CRYAB, LEDGF₍₁₋₃₂₆₎, and PHHP (P 1)-LEDGF₍₁₋₃₂₆₎ across the cornea layers of bovine and rabbit eyes.

FIG. 9 are graphs of quantitation of in vitro cell uptake study of FITC labeled PHIP (P 1) and TAT peptides in ARPE-19 cells. Cells were grown to a 10,000 well density in 96 well fluorescent imaging plates with a transparent bottom. The ARPE-19 cells required an additional 48 hrs, to reach this cell density. Cells were incubated with 200 μl of 10 and 5 μM/ml FITC labeled PHHP (P 1) and TAT peptides and uptake was continued for 1 hour at 37° C. Following uptake; cells were washed three times with acidic PBS (pH 5.0) and normal PBS (pH 7.4) at 37° C. Cells were lysed using 0.1 M NaOH and quantified using a fluorometry method. Panel A illustrates uptake of peptides by ARPE-19 cells and absolute amount, and panel B illustrates the percentage uptake of peptides at different concentrations. It also shows the 4-7 fold higher uptake of the PHHP (P 1) peptide compared to the TAT control peptide.

FIG. 10 is a graph illustrating uptake of PHHP (P 2) conjugated with Nile red loaded PLGA NPs by HUVEC cells.

FIG. 11 are a series of PHHP peptide (P 1 to P 6) models generated using free PEP-FOLD online software.

### DETAILED DESCRIPTION OF THE INVENTION

As used herein “peptides” refers to sequence of amino acids linked with peptide bonds in their natural or chemically modified form such as pegylation, acetylation, methylation, amidation, and hydroxylation for intended purposes.

As used herein “Pharmaceutical or biological use” refers to use of therapeutic agents and/or carriers for drug substances for the treatment of diseases.

As used herein “peptide nanobodies” refers to any structural compositions with regular or irregular shapes with particle sizes in the range about 1 nm to about 1000 nm.

As used herein “peptide-drug covalent conjugates” refers to physical or chemical crosslinking to or complexes with drug molecules, this includes peptides, proteins, nucleic acids, siRNA, miRNA, mRNA, gene-containing viral vectors, small drug molecules, carbohydrates, steroids and small molecule drugs alike used for therapeutic purposes.

As used herein “distinct primary and secondary” refers to the sequence of amino acid residues in a linear order in peptides and the three dimensional structure acquired by these linear arrangements in aqueous and non-aqueous solvents or mixtures thereof. The secondary structures could fall into categories of regular arrangements like helices, beta strands/sheets, loops and coils or they could have random structures.

As used herein “secondary structural domains” refers to regions in peptide which could form locally defined three-dimensional structure having unique biophysical and functional attributes like stability, free energy, binding characters, interactions with other molecular entities and activity.

As used herein “hydrophobic, hydrophilic, and amphipathic regions” refers to stretches of peptide which have unique solvation properties in in polar and non-polar solvents and a combination thereof.

As used herein “macromolecules” refers to any molecule having size larger than 1 kDa and having biological or non-biological source like proteins, peptides, nucleic acids, siRNAs, miRNAs, lipids, carbohydrates, polymers and alike.

As used herein “small molecules” refers to any molecule having size up to 1 kDa and having biological or non-biological sources with therapeutic or non-therapeutic applications.

As used herein “carriers” refers to entities used to carry, transport, delivery, migration, permeation, and translocation drug candidates and other entities to the systemic circulation, crossing tissues and cellular barriers, cell walls and membranes, cell organelles, and a like structure in biological systems.

Positive-helix-hydrophobic peptides (PHHP) are with an amphipathic beta strand-turn-alpha helix (βTα) motif domains. Subsequently, to simplify matters it is just referred to as the PHHP peptides or PHHP peptide variants. Peptides of the present invention contain custom designed amino residues. In one exemplary embodiment, these PHHP peptides comprises the unique linear sequence amino acids linked together by peptide bonds. These peptides can bind to cell membranes and facilitates entry of cargos associated to it either in physical or chemical conjugated forms across tissues and cellular boundaries.

In one other exemplary embodiment, these PHHP peptides were synthesized and characterized for their physicochemical properties using computational tools. These peptides were found to be basic in nature, positively charged under physiological conditions, and consist of distinct hydrophobic and hydrophilic domains within their structure.

In another exemplary embodiment, these PHHP peptides formed unique secondary structures required for their function. They can form various nano and micro structures under appropriate formulation conditions, such as; the presence of metal ions, salts, pH, detergents, surfactants, polar, and non-polar solvents. These nano and micro structures can encapsulate, complex, and conjugate with other small or macro molecules.

In one other exemplary embodiment, these PHHP peptides form molecular assemblies of various sizes under different formulation conditions. These assemblies can be nano-scale assemblies or micro-scale assemblies, and may include nanomicelles, nanoprecipitates, nanocomposite, nanodisc, nanorod, nanoparticles, solid nanoparticles, microparticles, vesicles, amorphous aggregates, and capsules. These nanoassemblies can be formed by a combination of various formulation conditions like salts, metal ions, surfactants, pH, and so on.

In certain exemplary embodiments, the nano- and micro-assemblies can be used to deliver various therapeutic agents like proteins, peptides, nucleic acids, plasmid DNA, siRNAs, miRNAs, mRNAs, gene-containing viral vectors, small molecules, and therapeutic agent containing nanoparticle carriers of various types across various cellular and tissue barriers. Alternatively, the PHHP peptides can be fused or otherwise joined with the above therapeutic agents or their carriers with or without an appropriate linker to enhance or sustain delivery.

In certain exemplary embodiments, these PHHP peptides can be complexed, conjugated, adsorbed, and linked to the nano- and micro-carriers made up of suitable polymeric and non-polymeric materials that may be used in the present invention including but not limited to; polylactide (PLA), poly (D,L-lactide-co-glycolide polymers (PLGAs), cellulose derivatives, chitosan, sugar based polymers, lipids, polyethylene (PE), polypropylene (PP), iron oxide, cerium oxide, zinc oxide, poly (tetrafluoroethylene), poly (ethyleneterephathalate), gold, silver, other biocompatible metals, crystals, and silica. These nano- and micro-carriers may encapsulate, complex, adsorb, therapeutic molecules.

In another exemplary embodiment, these PHHP peptides may be conjugated, fused, or otherwise joined with protein molecules using genetic engineering methods to produce fusion complexes in expression hosts of bacterial and eukaryotic origins. These fusion constructs with or without built-in linkers can directly be used deliver protein therapeutics across cellular and tissue barriers.

In an embodiment, the PHHP peptides composition comprises about 0.01 to about 5 mg/mL of individual or combinations of PHHP peptides in nanoassemblies formulations.

In one other exemplary embodiment, these PHHP peptides can be formulated individually or in combinations in concentration range on 0.01 to 5.0 mg/ml with various stabilizers such as aliphatic alcohol, fatty acid and a salt thereof, fatty acid ester, polyalcohol alkyl ether, glyceride, and organic amine.

In one other exemplary embodiment, these PHHP peptides can be formulated individually or in combinations with various amount of a hydrating agent such as hyaluronic acid, and/or polyvinylpyrrolidone, may be included in any of the compositions of the invention for enhanced retention at site of delivery.

The stabilizers and hydrating agent can be used in the range of 0.1 to 5% by weight of the composition.

In one other exemplary embodiment, surfactant may be included in the compositions of the invention to facilitate dissolution of PHHP peptide components and facilitate the formation and stabilization of nanoassemblies and cargo molecules (such as protein, peptides, nucleic acids, and small molecules) adsorbed or chemically cross-linked.

In another embodiment surfactant can be used individually or in combination from the group of anionic surfactant, cationic surfactant, nonionic surfactant and amphoteric surfactant to stabilize the PHHP delivery systems. Useful surfactants include fatty acid salt, alkyl sulfate, polyoxyethylene alkyl sulfate, alkyl sulfo carboxylate, alkyl ether carboxylate, amine salt, quanternary ammonium salt, polysorbate 80, and poloxamers. These surfactants can be used in the ranges of 0.05 to 1% by weight of the composition.

A pH adjuster may be used in the compositions to adjust pH of the composition to a desired range, such as pH 4-10, or pH 5-8, for example or any range that maximizes the permeation and transcellular delivery of nanoassemblies through the various surface or systemic barriers and stabilize the formulation.

In another embodiment various stabilizers can be used in combination with above agents to stabilize protein and nucleic acid cargos. Examples of such stabilizers includes glycerol, polyethylene glycol, sorbitol, mannitol, propylene-glycol, 1,3-butanediol, and trehalose. These stabilizers can be used individually or in combination in the ranges of 0.5 to 10% by weight of the composition.

### Examples

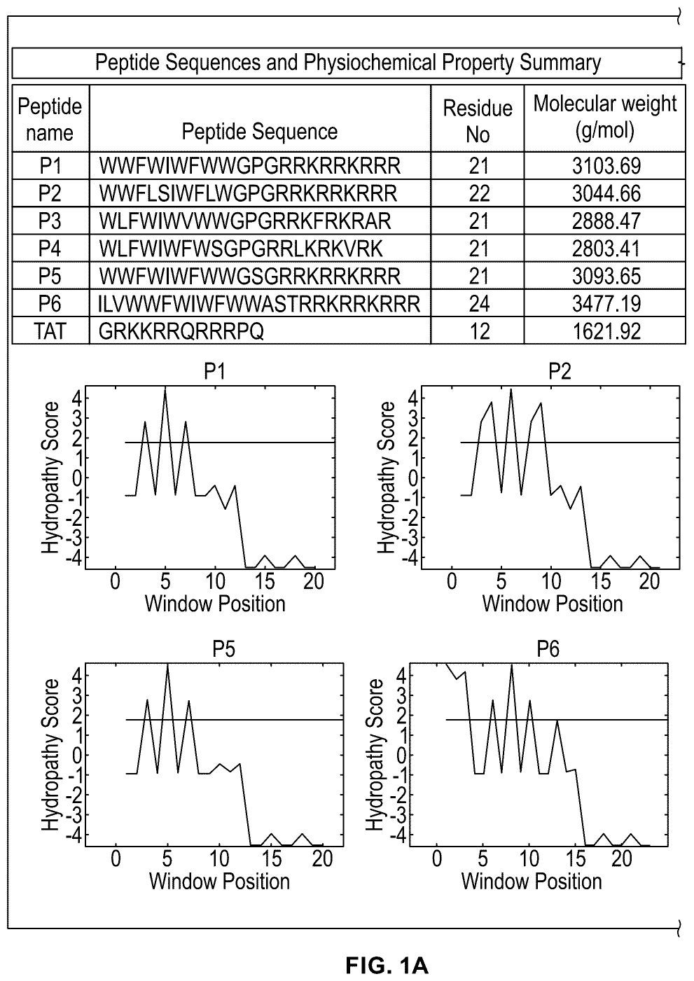

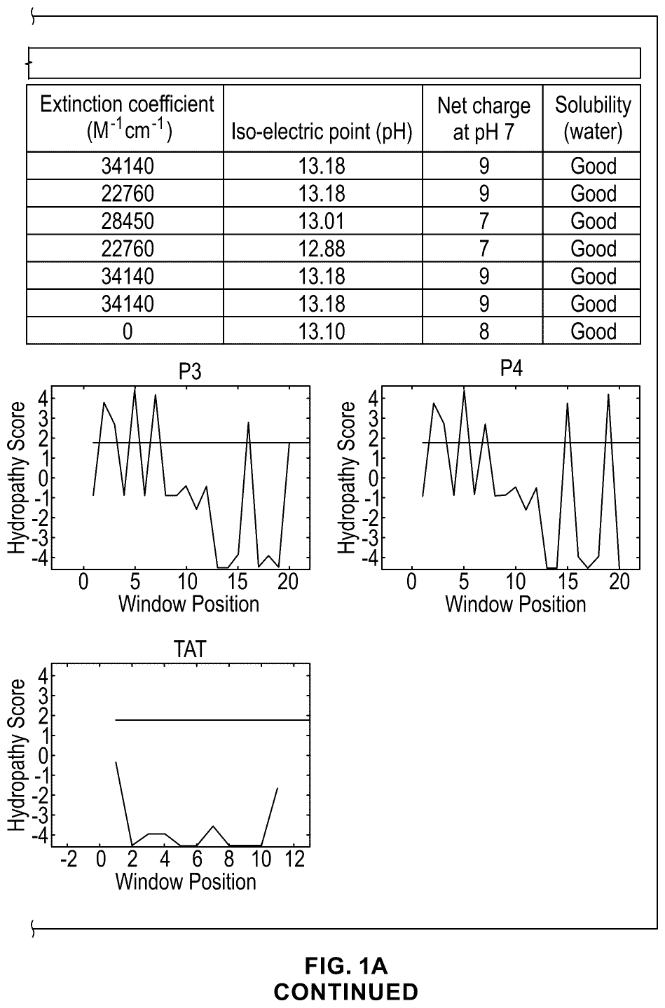

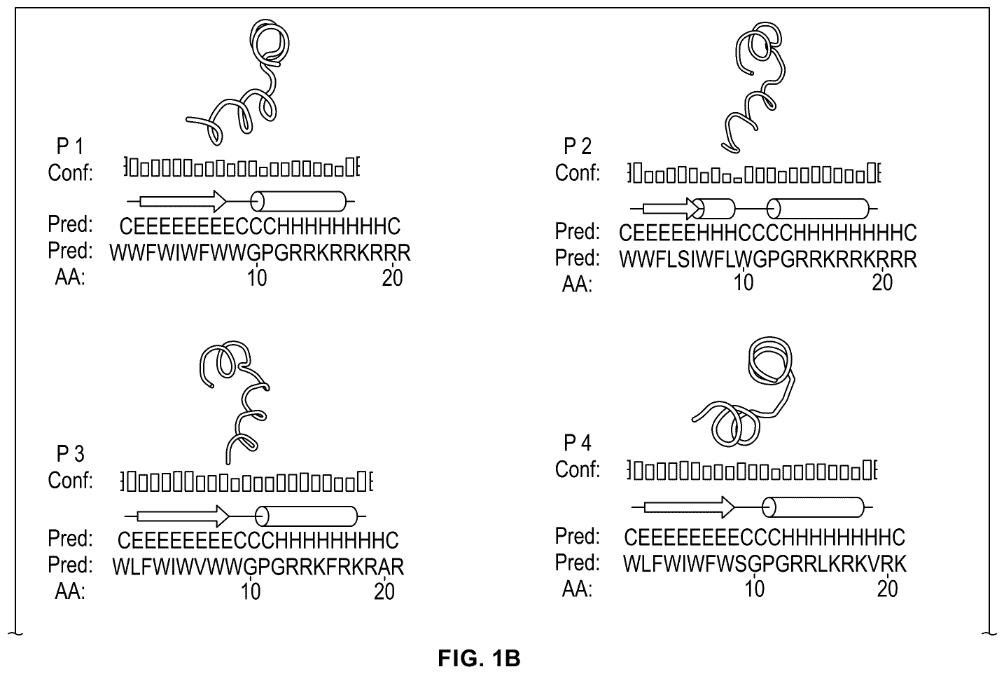

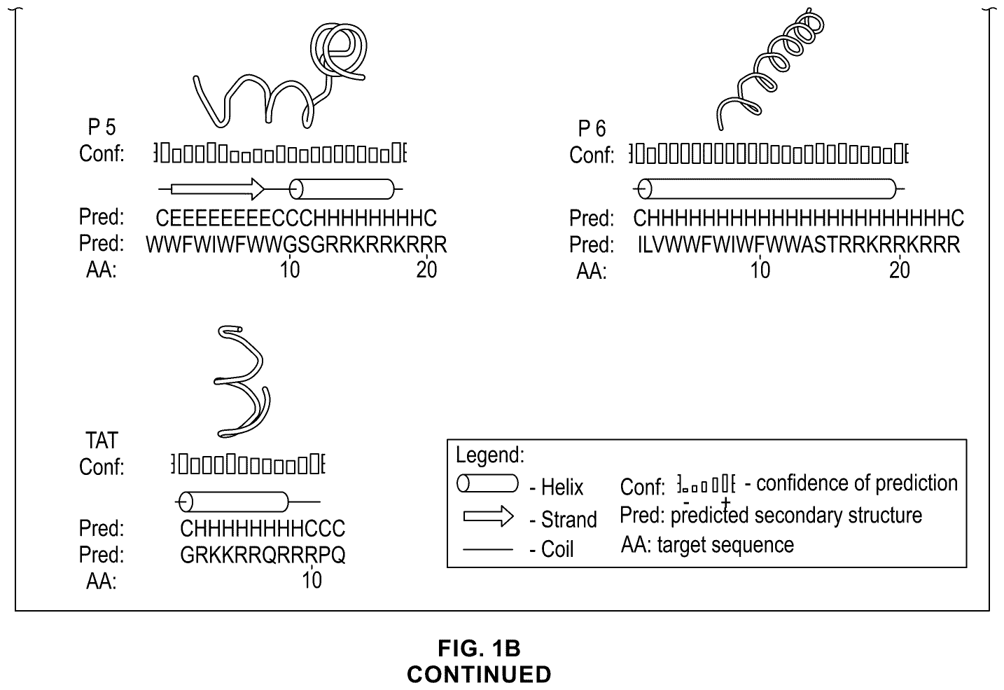

PHHP peptides were designed and synthesized. They were characterized based on their amino acid sequence and molecular weight using mass spectrometry. Peptide purity was analyzed by using a reverse phase HPLC method. The sequences and physicochemical characteristics of PFHHP peptides of the present inventions have been included in FIGS. 1A and B.

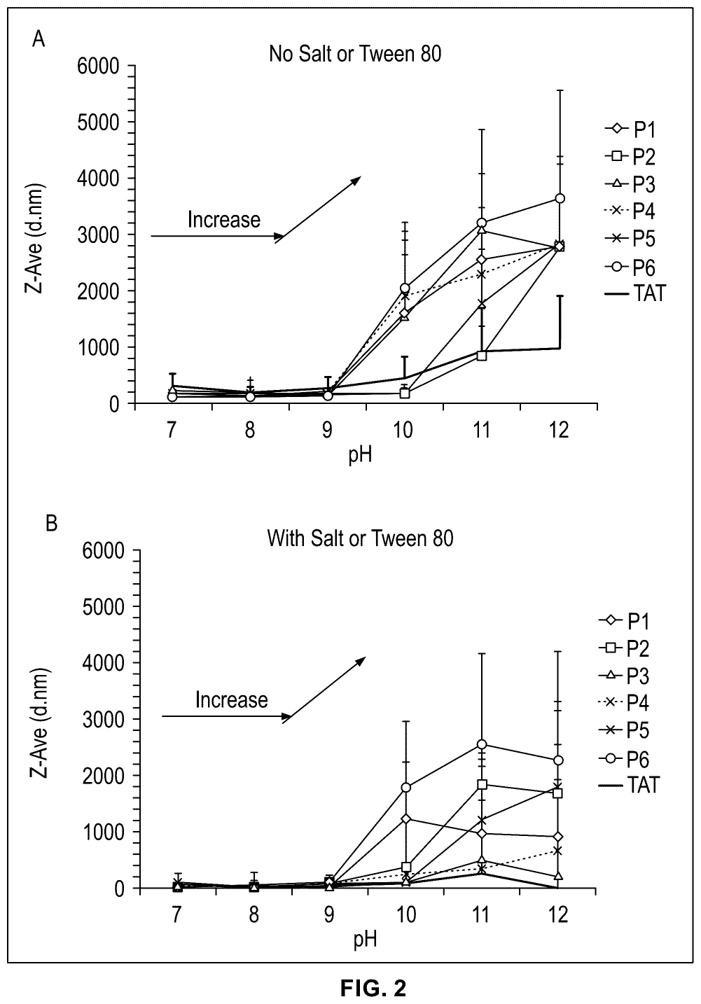

The effect of salt, pH, and buffers on size and zeta potential of nanoassemblies was determined. The peptides were incubated in buffers with different pH values and their size and zeta potentials were measured using a Malvern Nano-ZS instrument. The PHHP (P 1) and TAT peptides with concentrations of 10 μM were prepared in 50 mM Tris-HCl at pH 7.0 to 12.0. Samples were incubated at 25° C. for 1 hour and the size was determined using the Malvern Nano-ZS at 25° C. (the size test consisted of 11 runs per sample with a duration of 10 seconds per run). Similarly, these samples were also prepared in the previously mentioned buffer consisting of 0.5 M NaCl, and 0.1% Tween-80 (Polysorbate 80) surfactant and size was again determined following 1 hour incubation at 25° C. This data is presented in FIG. 2.

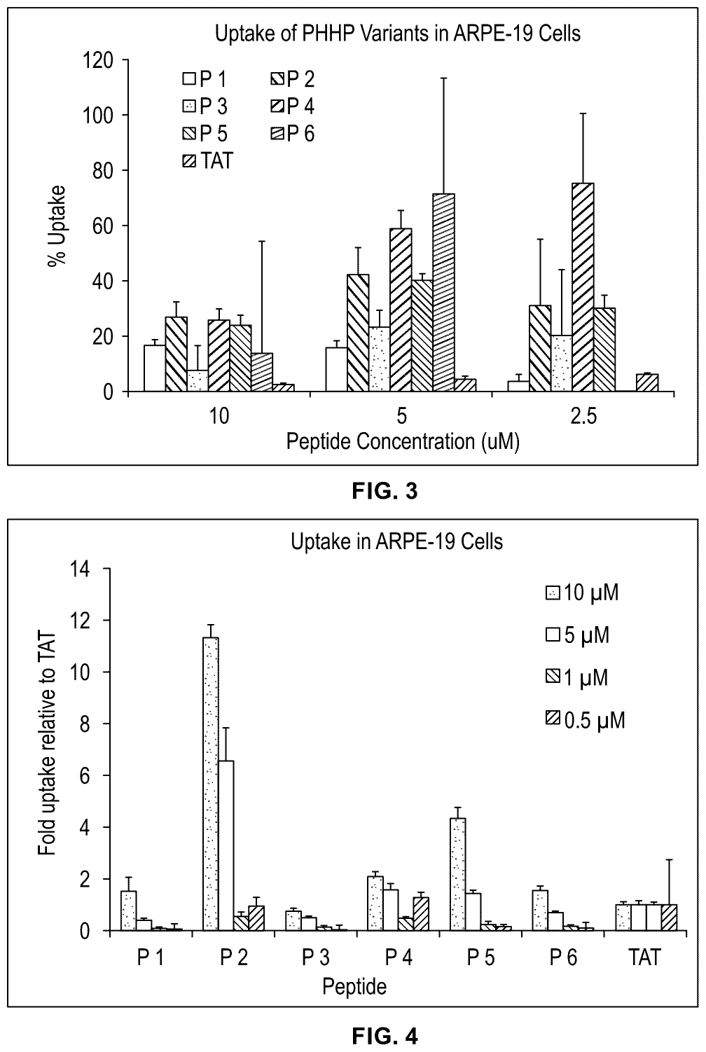

The cell uptake study of FITC labeled PHHP peptides in an ARPE-19 cell line was studied using fluorometry. ARPE —19 cells were grown in a DMEM/F12 medium. A cell density of 10,000 cells was plated into a 96 well plate for each cell lines, 48 hours before the start of the uptake study. The FITC labeled peptides with the following concentrations; 10 μM, 5 μM, 2.5 μM, and 1 μM were added to the wells in triplicates (200 μl). Following 1 hour of uptake, cells were washed three times with acidic PBS (pH 5.0) and normal PBS (pH 7.4). Cells were incubated with 200 μl of 0.1 M NaOH at 4° C. for 1 hour for lyses. Similarly, standards were prepared for each of the peptides by adding 200 μl of peptide solution in NaOH to un-incubated washed cells with different concentrations from 0.1 μM to 5 μM. All the samples were brought to room temperature and fluorescence spectra were recorded at an excitation wavelength of 493 nm and emission wavelength of 520 nm. The percentage uptake and absolute uptake were determined for each peptide at different concentrations and plotted as a bar graph for comparative analyses in FIG. 3.

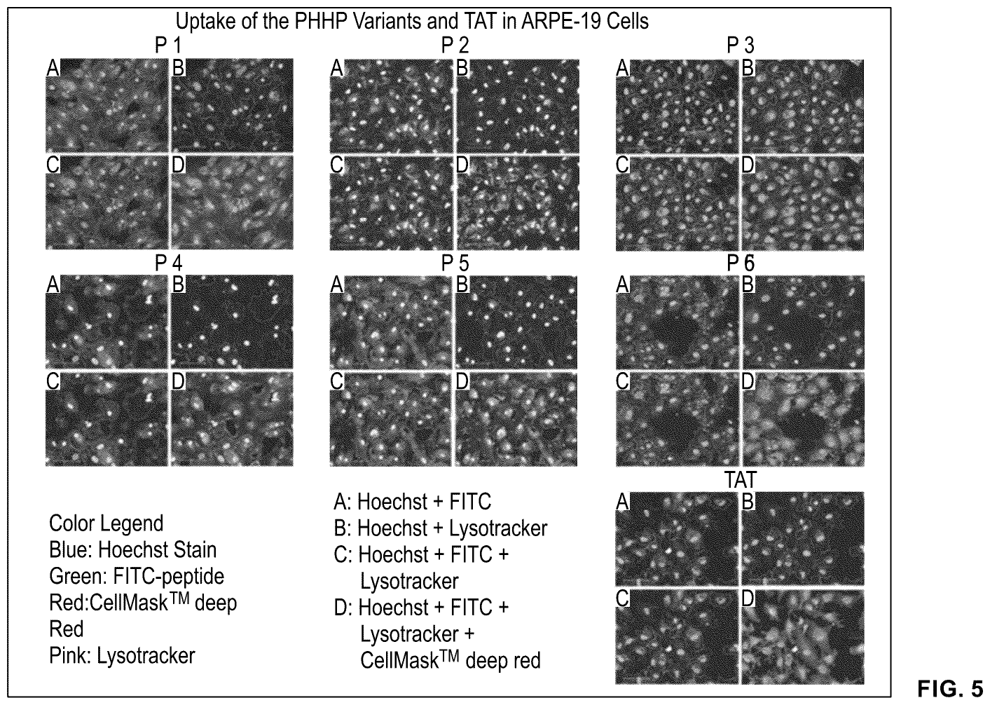

The cell uptake study of FITC labeled PHHP peptides in an ARPE-19 cell line was studied using a fluorescence imaging method. ARPE-19 cells grown in DMEM/F12 medium. 5,000 cells were plated into the wells of a 96 well plate for each of the cell lines 18 hours prior to the start of study. The FITC labeled peptides with the following concentrations; 10 μM, 5 μM, 1 μM, and 0.5 μM were added to the wells in triplicates (200 μl). Following 1 hour of uptake, cells were washed three times with PBS (pH 7.4). Then cells were stained with Hoechst nuclear stain (1 μg/ml) in media and 100 μl of it was added into each well and incubated for 15 min. at 37° C. Similar cells were incubated with 100 μl of CellMask™ red membrane stain (1 μg/ml) and Lysotracker stain (0.1 μM) in respective media and stained for 5 minutes at 37° C. Following staining cells were washed three times with PBS. Then, cells were fixed with 3.7% paraformaldehyde (50 μl/well) for 20 minutes at room temperature. Cells were again washed three times with PBS and covered with 200 μl of PBS before being imaged under the fluorescence microscope (FIGS. 4 and 5).

Design and cloning of human lens epithelium derived growth factors fragment (LEDGF₍₁₋₃₂₆₎), human alpha crystallin B (CRYAB), and their fusion constructs with PHHP 1. Human lens epithelium derived growth factor fragment (LEDGF₍₁₋₃₂₆₎) was amplified from full length construct using primers (Forward Primer: 5′AGCAAGCCATGGGC ATGACTCGCGA TTTCA AACCTGGA3′[SEQ ID 7]; Reverse Primer: 5′ AGCAAGAAGCTTCTACTGCTCAGTTT CCATTTGTTCCTC3′ [SEQ ID 8]). Following PCR amplification, this construct was cloned in pET28a-c(+) vector at NcoI and Hind III restriction sites, without a polyhistidine tag. The PHHP 1) N-terminal fusion construct of (LEDGF₁₋₃₂₆) was generated by amplification of (LEDGF₍₁₋₃₂₆₎) gene construct in pET28a-c(+) using a special large forward primer

(5′agcaagccatgggcTGGTGGTTTTG GATTTGGTTTTGGTGGGGCCC
GGGCCGCCGCAAACGCCGCAAACGCCGCCGCatgactcgcgatttcaaac
ctgga3′ [SEQ ID 9])

consisting of bases to code for a PHHP 1 with following sequence WWF WIWFWWGPGRRKRRKRRR [SEQ ID 1]. The PHHP 1 was designed based on the biophysical and physicochemical properties to have a greater role in cell membrane binding and in transient alternation of the plasma membrane fluidity. Similarly, human alpha crystallin B gene was amplified from human lens epithelium cDNA library using following primers (Forward primer: 5′aagctgccatggacatcgccatccaccaccc3′ [SEQ ID 11]; and Reverse primer: 5′gagagacatatgctatttctt gggggctgcggtgac 3′[SEQ ID 12]). Following PCR amplification, this construct was cloned in pET28a-c(+) vector at NcoI and NdeI restriction sites, without a histidine tag. A PHHP 1 containing gene was fused to the N-terminal of CRYAB gene by amplification of CRYAB gene construct in pET28a(+) using a modified forward primer (5′aagctgccatgggcTGGTGGTTTTGGATTTGGTTTTGGTGGGGCCCGGGCCGCCGCAAAC GCCGCAAACGCCGCCGC atggacatcgccatccaccaccc3′ [SEQ ID 10]) consisting of bases to code for a PHHP 1 with the following sequence, WWFWIWFWWGPGRR KRRKRRR [SEQ ID 1]. In both the cases PHHP 1 fused constructs were digested with respective restriction enzymes (NcoI, HindIII, and NdeI) and cloned in pET28a-c(+) vector. After ligation, all the constructs were used to transform E. Coli DH5a bacteria strains. Transformed colonies (10) were picked for each construct and grown overnight in 5 ml of LB medium in presence of 25 μg/ml kanamycin at 37° C. and 220 rpm. Plasmids were isolated from an overnight culture using a Qiagen plasmid mini-prep isolation kit and the DNA concentration was determined at 260 nm. Isolated plasmids were PCR amplified and double digested with respective restriction enzymes and analyzed on agarose gel to determine the presence of LEDGF₍₁₋₃₂₆₎, PHHP 1-LEDGF₍₁₋₃₂₆₎, CRYAB and, PHHP 1-CRYAB gene inserts in a recombinant vector. Afterward, these isolated plasmids were also sequenced using T7 promoter primer and sequences were analyzed to check reading frame and changes in base sequences during cloning process. Once clones were identified based on right sequence and reading frames, they were used to transform E. coli expression host strain BL21 (DE3) for protein biosynthesis. Cloning of CRYAB, LEDGF₍₁₋₃₂₆₎, and their PHHP peptide variants (P1 to P 6) was carried out in high expression vector, pET28a-c(+). Primers were designed for wild type CRYAB, and LEDGF₁₋₃₂₆ and amplified and purified using gel extraction methods. Similarly, primers having codon optimized sequences for PHHP 1 peptide were also designed and used to fuse it to N-terminal of CRYAB and LEDGF gene fragment. Amplified fragments were analyzed on agarose gel showed fusion and amplification PHHP 1 fused CRYAB and LEDGF gene fragment. PCR amplified constructs were digested with NcoI and NdeI in case of CRYAB, and PHHP 1-CRYAB and with NcoI and HindIII in case LEDGF gene fragment and PHHP 1-LEDGF gene fragment and ligated in pET28a-c(+) vector. A Ligation reaction was used to transform E. coli DH5a strain and isolated plasmids were used to check the insertion of gene constructs using PCR restriction digestion and sequencing methods. PCR amplification and restriction digestion of purified recombinant plasmids showed presence of respective gene constructs in pET28a-c(+) vector. Analyses of cloned gene constructs sequences of CRYAB, PHHP 1-CRYAB, LEDGF gene fragment, and PHHP 1-LEDGF gene fragment showed their insertion in right reading frame compatible with expression and translation frame.

Expression and purification of lens epithelium derived growth factors fragment (LEDGF₍₁₋₃₂₆₎), human alpha crystallin B (CRYAB), and their PHHP 1 fusion constructs. E. coli strain BL21 (DE3) transformed with human lens epithelium derived growth factors fragment (LEDGF₍₁₋₃₂₆₎), human alpha crystallin B (CRYAB), and their PHHP 1 fusion constructs were used for protein expression, and isolation. First, recombinant clones were grown, as primary culture in LB medium (5 ml) in the presence of appropriate antibiotics, overnight at 37° C. and 220 rpm. Cultures grown overnight were used as an inoculum and transferred to Erlenmeyer flask with 100 ml medium (at 1% inoculum concentration), and grown to reach an optical density (OD) of 600 nm at 0.8-1.0. At this point, the culture was induced by adding IPTG at 1 mM final concentration and grown for another 4 hours at 37° C. Cells were harvested by centrifuging the culture at 8,000 g for 10 minutes at 4° C. Protein expression was checked by using SDS-PAGE of induced and non-induced cell cultures. Expression of PHHP 1-LEDGF₍₁₋₃₂₆₎, and PHHP 1-CRYAB was carried out by inducing the culture with 1 mM IPTG overnight at 25° C. and 220 rpm. For purification of recombinant proteins, E. coli cells expressing the protein of interest were grown in and harvested from 1 liter LB culture. All the recombinant protein expressed were purified using a two-step chromatography process, which involved cation exchange followed by gel filtration chromatography. Cells expressing LEDGF₍₁₋₃₂₆₎ were harvested and re-suspended in Tris-HCl buffer (25 mM Tris-HCl, 1 mM EDTA, pH 7.0) consisting of protease inhibitor cocktail and sonicated for 10 minutes (pulse 10 sec with 10 sec gap, power output 24 W) under ice cold conditions. Lysed cells were centrifuged at 25,000 g for 30 minutes to separate supernatant and pellet fractions. Supernatant fraction was subjected to SP Sepharose ion-exchange chromatography. LEDGF₍₁₋₃₂₆₎ and bound proteins were eluted by using sodium chloride continuous gradient from 0 to 1.0 M NaCl, and fractions (10 ml) were collected analyzed on SDS-PAGE. Fractions containing LEDGF₍₁₋₃₂₆₎ were pooled separately, dialyzed three times against Tris-HCl buffer (25 mM) containing 100 mM NaCl and 1% sucrose and lyophilized. The lyophilized protein was reconstituted in water and further purified using Superdex® 5-200 gel filtration chromatography. Similar process was used for purification of PHHP 1-LEDGF₍₁₋₃₂₆₎ protein. CRYAB and PHHP 1-CRYAB expressed cells were harvested (1 liter culture) and cell pellets were re-suspended in 30 ml of Tris-HCl buffer, 50 mM; 1 mM EDTA; 5% sucrose, pH 8.0 consisting protease inhibitor cocktail and sonicated for 10 min (10 sec pulses with 10 sec gaps, power output 24W) under ice cold conditions. Lysed cells were centrifuged at 25,000 g for 30 minutes to separate supernatant and pellet fractions. Supernatant fraction were subjected to DEAE ion-exchange chromatography, and PHHP 1-CRYAB, CRYAB, bound proteins were eluted using sodium chloride continuous gradient elution from 0 to 1.0 M NaCl. Fractions (10 ml) were collected and analyzed using SDS-PAGE. Fractions containing PHHP 1-CRYAB and CRYAB pooled separately, dialyzed and further purified using Superdex® S-200 gel filtration chromatography as mentioned above. All the above gel filtration fractions consisting of pure proteins were pooled, dialyzed against phosphate buffer (10 mM, pH 7.4) consisting of 100 mM NaCl and 5% sucrose, then they were stored at −80° C. pending characterization. CRYAB and LEDGF₍₁₋₃₂₆₎ expression in the E. coli BL21 (DE3) host was optimized for IPTG concentration, temperature, and post-induction time. It was observed that 1 mM IPTG at 37° C. and 220 rpm, with a 4 hour post-induction period were the optimal conditions for high level expression. While overnight induction with 1 mM IPTG at 25° C. was found to be optimal for PHHP 1 variants of CRYAB and LEDGF₍₁₋₃₂₆₎. All the above proteins were expressed in the soluble forms as evident from the SDS-PAGE of lysed cell supernatant and pellet sample fractions. Because the Isoelectric point (pI) of CRYAB protein in the acidic range, its purification was carried out using DEAE-Sepharose anion exchange chromatography while the other proteins were purified using SP-Sepharose cation exchange chromatography. Bound proteins were eluted using a sodium chloride gradient where CRYAB and LEDGF₍₁₋₃₂₆₎ eluted at lower NaCl concentration while the PHHP 1 variants eluted at higher concentration of NaCl owing to their higher basic isoelectric points. Ion-exchange fractions consisting of the partially impure form of CRYAB, LEDGF₍₁₋₃₂₆₎ and their PHHP 1 variants were pooled individually, dialyzed, concentrated and further purified using Superdex® S-200 sephacryl gel filtration chromatography and pure fractions were pooled and analyzed on SDS-PAGE. The CRYAB, LEDGF₍₁₋₃₂₆₎, and their PHHP 1 variants were purified to homogeneity using the previously mentioned methods and pure proteins were analyzed on SDS-PAGE which showed their pure form in purified fractions.

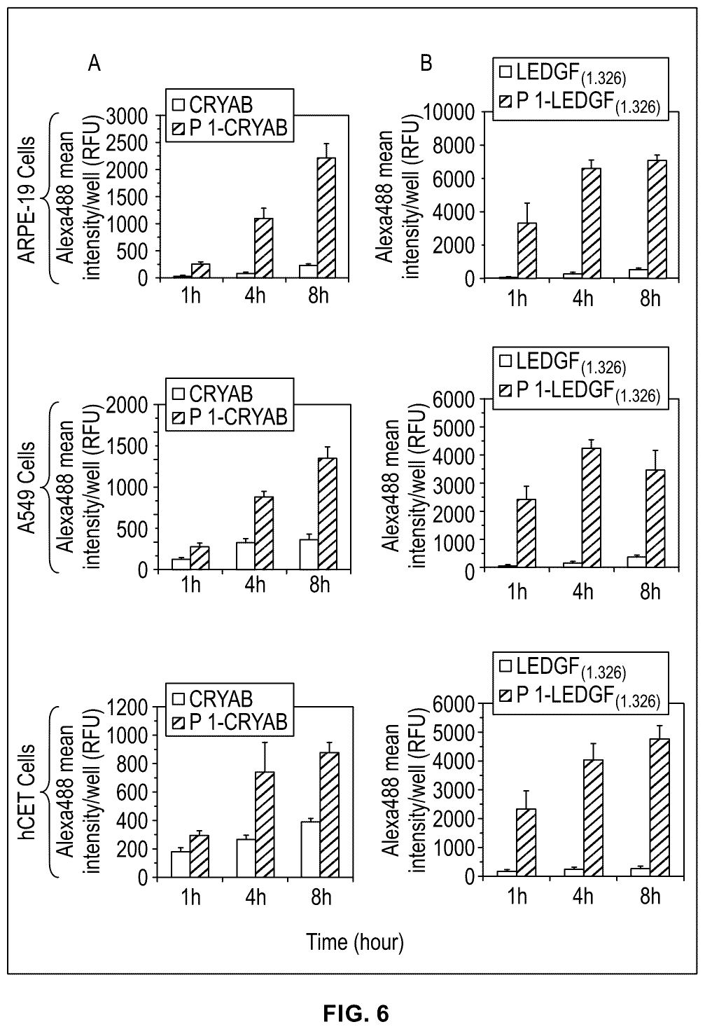

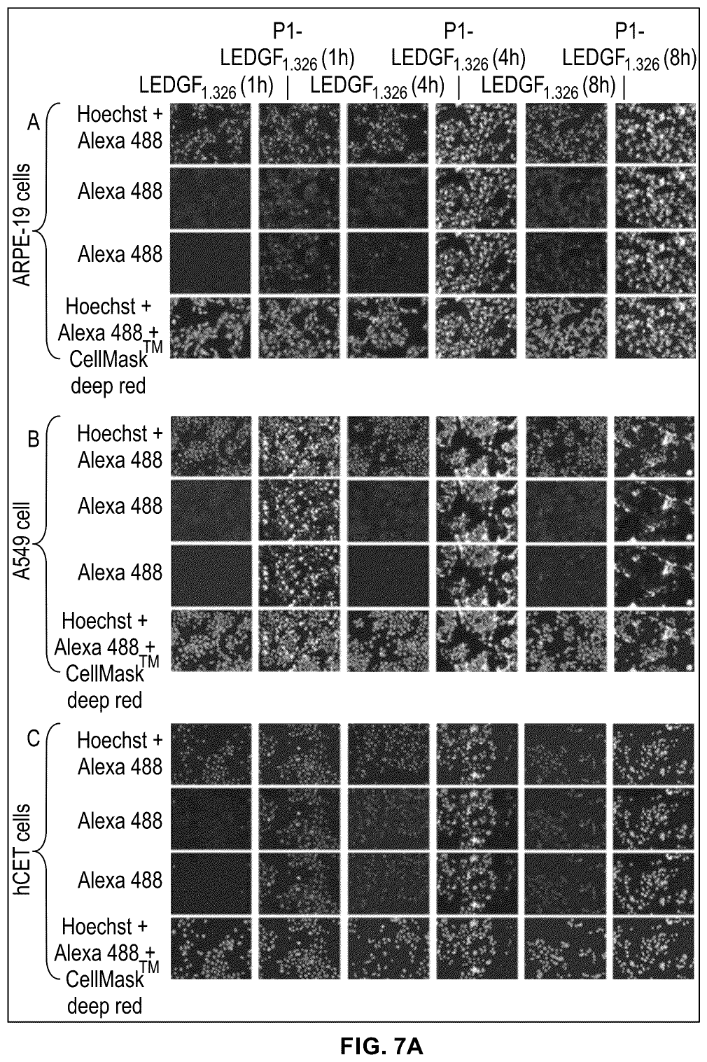

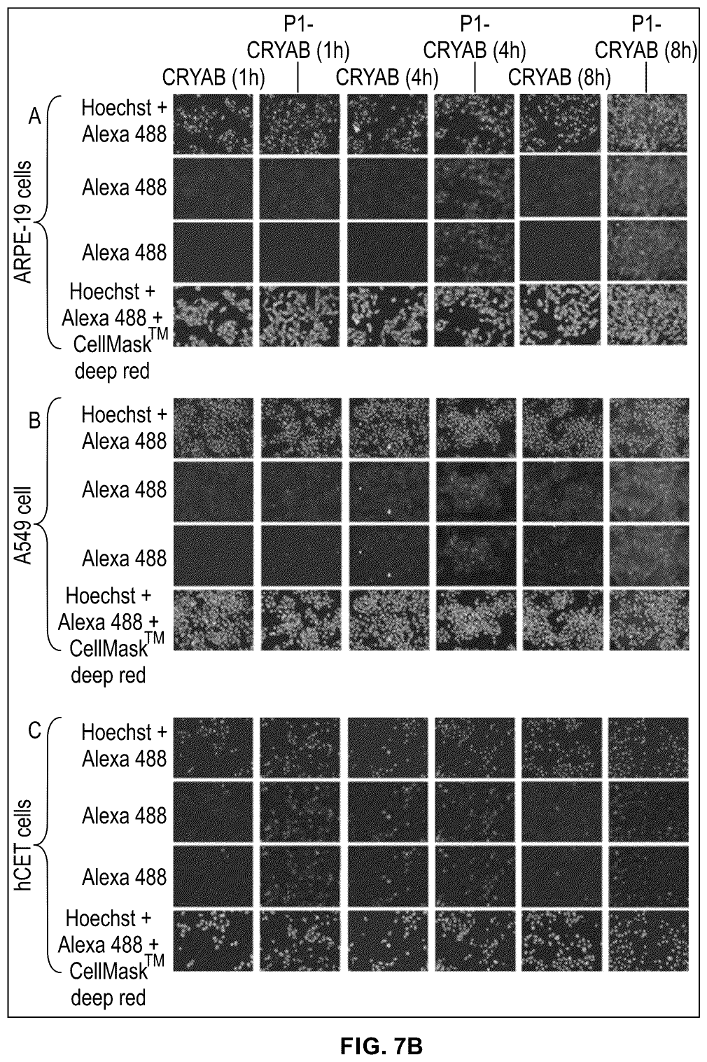

In vitro cell uptake study of lens epithelium derived growth factors fragment (LEDGF₍₁₋₃₂₆₎), human alpha crystallin B (CRYAB), and their PHHP 1 fusion constructs in ARPE-19, A-549, and hCET cell lines. Cell uptake studies were carried out using Alexa Fluor® 488 labeled proteins. Recombinant proteins (LEDGF₍₁₋₃₂₆₎, PHHP 1-LEDGF₍₁₋₃₂₆₎, CRYAB, and PHHP 1-CRYAB) were estimated for protein concentration using micro-BCA assay kit. 1 mg of each protein was labeled with Alexa Fluor® 488 dye according to supplier instructions (Invitrogen). Alexa Fluor® 488 labeled proteins was dialyzed against PBS buffer (three times) to remove excess dye. Following dialysis, Alexa Fluor® 488 conjugated proteins were estimated for conjugation efficacy and protein content. The LEDGF₍₁₋₃₂₆₎, and PHHP 1-LEDGF₍₁₋₃₂₆₎ showed similar Alexa Fluor® 488 intensity at equal protein concentration. This was because of no significant difference in number of lysine amino acids where Alexa Fluor® 488 reacts with amino group (54 for LEDGF₍₁₋₃₂₆₎, and 56 in PHHP 1-LEDGF₍₁₋₃₂₆₎). A similar trend was observed for the CRYAB and PHHP 1-CRYAB (10 lysine's in CRYAB, and 12 in PHHP 1-CRYAB peptides). For the cell uptake study ARPE-19, A549, and hCET cells were grown in DMEM/F12, RPMI, and KBM epithelial cell media, respectively. In each well of a 96 well plate, 3,000 cells were plated and grown for 18 hours for the ARPE-19 and A-549 cells, whereas 48 hours was required for the hCET cells. In each well, 200 μl of Alexa Fluor® 488 labeled proteins (10 μg/ml) were added in triplicate and uptake was pursued for 1, 4, and 8 hour duration. Following uptake, cells were washed three times with PBS pH 7.4. Subsequently, cells were incubated with 100 μl of nuclear stain (1 μg/ml) in respective media and incubated for 15 min at 37° C. Further, cells were incubated with 100 μl of CellMask™ red membrane stain (1 μg/ml) in respective media and stained for 5 min at 37° C. Following staining, cells were washed three times with PBS at 37° C. Subsequently, cells were fixed with 3.7% paraformaldehyde (50 μl/well) for 20 minutes at room temperature. Cells were again washed three times with PBS at 37° C. and finally 200 μl of PBS was added to each well before being imaged using the Operetta® High Content Imaging System (Perkin Elmer, Waltham, MA). Images were acquired at 20× magnification, in four channels (Alexa Fluor® 488, DAPI, CellMask™ red, and bright field), the focus was at the center of cell nucleus. Images were processed for Alexa Fluor® 488 signal in each cell after base signal correction. For this purpose cells were identified by DAPI (blue) stain and cell boundary was defined using red membrane stain. Alexa Fluor® 488 conjugated protein signal was determined within cell boundary using the Harmony software (Version 3.1) for relative quantitation of protein uptake within groups (FIGS. 6 and 7).

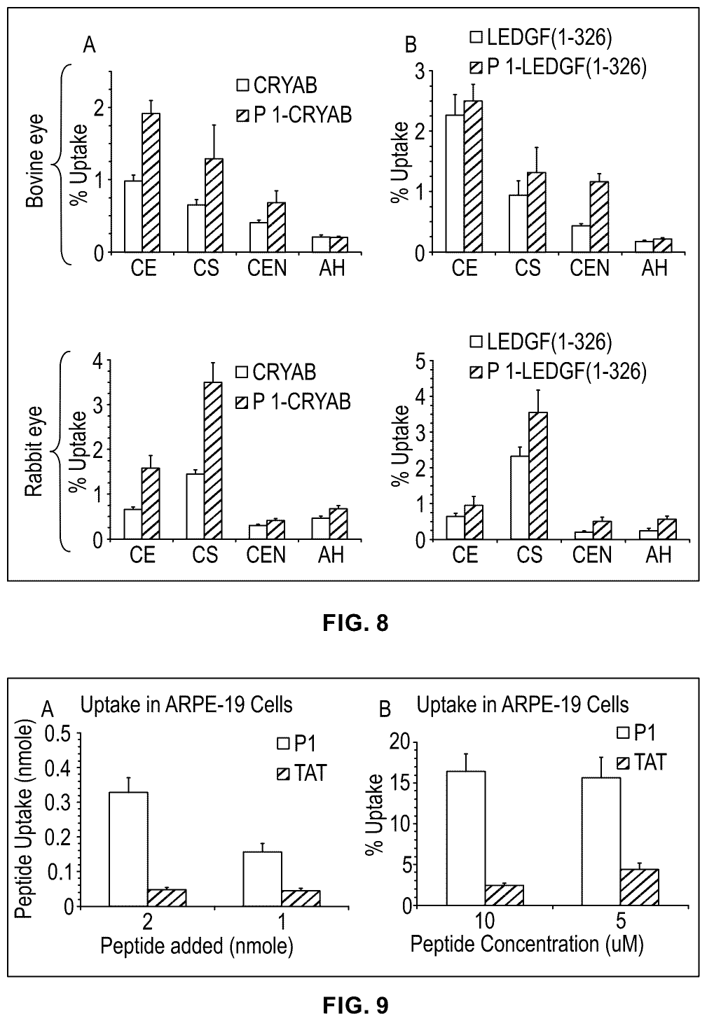

Ex vivo uptake and tissue distribution of lens epithelium derived growth factors fragment (LEDGF₍₁₋₃₂₆₎), and human alpha crystallin B (CRYAB) with and without PHHP 1 were determine across bovine and rabbit eyes. Fresh bovine eyes were received from a slaughter house. For the uptake study, eye balls were placed in muffin plates so that the posterior parts were submerged in assay buffer (pH 7.4 at 37° C.) and equilibrated for 30 minutes at 37° C. Alexa Fluor® 488 labeled LEDGF₍₁₋₃₂₆₎, PHHP 1-LEDGF₍₁₋₃₂₆₎, CRYAB, and PHHP 1-CRYAB proteins (20 μg/ml) in assay buffer were applied topically (50 l) on cornea surface (n=4 eyes) and uptake was continued at 37° C. for 5 min. The cornea tissue was kept moist by topical application of assay buffer (25 μl) at 1 minute intervals during the time course of uptake study. Evaporation from the eye surface was minimized by covering the muffin plates with Saran wrap. At the end of a 5 minute uptake period, the cornea was washed three times with acidic PBS (pH 5.0) followed by three washes with sterile PBS, pH 7.4 (1 ml) at 37° C. Different corneal layers including epithelium, stroma, and endothelium, and aqueous humor were isolated and Alexa Fluor® 488 labeled proteins were extracted from the tissues by using homogenization (3,000 rpm for 1 min) and bath sonication (15 min. at 37° C.) methods in 0.5 ml of lysis buffer (50 mM Tris-HCl, 100 mM NaCl, 2% Triton X-100, pH 7.4) consisting of 1× protease inhibitor cocktail. Proteins from the samples and standards were quantified using fluorometry method. Tissue levels of Alexa Fluor® 488 labeled proteins were determined and plotted as % uptake relative to the topical dose applied. A similar study was carried out with rabbit eyes (Pel-Freez, Rogers, AR) where all the processes were similar except the amount of topical dose was 30 μl. Rabbit eyes were kept moist with assay buffer at 5 minute intervals (FIG. 8).

Intracellular uptake of TAT and PHHP 1 peptides in ARPE-19 cells. ARPE-19 cells (10,000 cells/well) were plated in 96 well plate and grown in DMEM/F12 medium for 48 hours prior to the start of uptake study. Different concentrations (10 μM, and 5 μM) of FITC labeled PHHP 1 and TAT peptides were added in triplicates (200 μl) in the wells. Following 1 hour of uptake at 37° C., cells were washed three times with acidic PBS (pH 5.0) and normal PBS (pH 7.4). Cells were lysed by incubating with 200 μl of 0.1 M NaOH at 4° C. for 1 hour. Similarly, calibration curves for each peptide were prepared by adding 200 μl of solution of 0.1 μM to 5 μM of each peptide in 0.1 M NaOH to the untreated cells. All the samples were then brought to room temperature and fluorescence intensities were recorded at an excitation wavelength of 493 nm and emission wavelength of 520 nm.

Percentage uptake and statistical analyses at different concentrations were compared. The percentage uptake of FITC labeled PHHP 1 and TAT peptide in ARPE-19 cells, exhibited a concentration dependence in uptake behavior. The fold improvement in the uptake of PHHP 1 peptide was ˜4-7 relative to the TAT peptide. The percentage uptake of the PHHP 1 peptide was determined to be ˜15-17, whereas ˜3-4% for TAT peptide. This uptake study lead us to create PHHP 1 fusion constructs of two therapeutic proteins (LEDGF₍₁₋₃₂₆₎ and crystallin B variant) using recombinant DNA technology and their purification from E. coli cells lysate following recombinant expression and isolation. Furthermore, these proteins in their PHHP 1 tagged and un-tagged forms were evaluated for intracellular uptake properties in ARPE-19, A549, and hCET cells lines and for transcellular migration behavior across bovine and rabbit eyes cornea following topical dosing (FIG. 9).

Uptake of Nile red loaded PLGA NPs were studied in HUVEC cell following surface conjugation with PHHP 2 peptide and compared with control PLGA NPs. Nanoparticles were prepared using a solvent evaporation method. 120 mg of PLGA polymer was weighed and dissolved in 3.6 ml of DCM in 2 separate glass vials. 6 mg Nile red was weighed and dissolved in 2 ml of DCM. 2% PVA solution was made in water and filtered. 0.4 ml of 3 mg/ml Nile red solution was added to 3.6 ml of polymer solution and mixed well. Next, this polymer solution was sonicated for 2 minutes at an amplitude of 40 in an ice bath after adding 0.8 ml of water.

Afterward, this primary emulsion was transferred to 20 ml 2% PVA solution and sonicated for 4 min at 80 amplitude (30 sec pulse on and off time). Then, the prepared final emulsion was stirred at room temperature for 6 hours to evaporate the DCM. Formed NPs were separated by centrifugation at 32,000 g for 30 minutes and further washed twice with water to remove any PVA. After washing the NPs were suspended in 10 ml water, snap frozen in liquid N2 and kept for lyophilization. For conjugation of PHHP 2 with PLGA NPs, 10 mg of NPs were weighed and dispersed in 1 ml of MES buffer containing 100 mM EDC and 200 mM NHS. PLGA NPs were activated for 2 hours at room temperature. Following activation, samples were centrifuged at 20,000 g for 30 minutes and the pellet was suspended in 1 ml PBS buffer containing 0.5 mg of FITC labeled PHHP-2. The reaction was continued for 3 hours to allow for peptide conjugation.

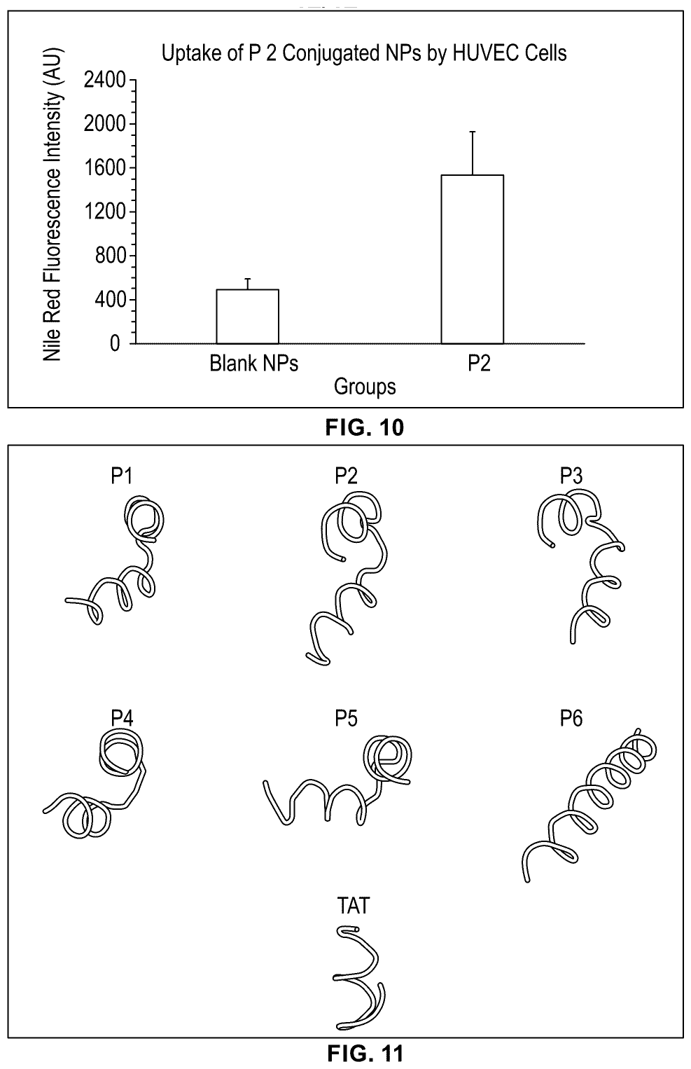

Following the conjugation reaction the mixture was centrifuged at 20,000 g for 30 minutes to separate functionalized NPs from free peptide. Then the NPs pellet were washed twice with water to remove buffer and free peptide. Next, the NPs pellet was suspended in 1 ml water and snap frozen in liquid N2 and lyophilized. For uptake study of NPs in HUVEC cells, 10,000 HUVEC cells were plated in a 96 well plate for 24 hours before the start of uptake study. The next day, cells were washed once with PBS (pH 7.4). 200 μl of NPs samples (Control and PHHP2-NPs) were prepared in serum free medium and added to wells. After 3 hours of uptake, cells were washed once with acidic PBS (pH 5.0) and twice with normal PBS (pH 7.4, under ice cold conditions) (PBS; 8 g/L NaCl, 0.2 g/L KCl, 1.44 g/L Na2HPO4, and 0.24 g/L KH2PO4 in water at pH 7.4). Following washing, cells were lysed in RIPA buffer and fluorescence was measured at an excitation wavelength of 560 nm and emission wavelength of 640 nm for Nile red (Figure. 10).

1. A positive, helical, and hydrophobic peptide (PFHHP) comprising at least one amphipathic beta strand-turn-alpha helix (βTα) motif domain.

2. The PHHP peptide of claim 1, having one of the following amino acid sequences or substantially homologous sequences having cell and tissue penetration properties: PHHP-1: WWFWIWFWWGPGRRKRRKRRR [SEQ ID 1]; PHHP-2: WWFLSIWFLWGPGRRKRRKRRR [SEQ ID 2]; PHHP-3: WLFWIWVWWGPGRRKFRKRAR [SEQ ID 3]; PHHP-4: WLFWIWFWSGPGRRLKRKVRK [SEQ ID 4]; PHHP-5: WWFWIWFWWGSGRRKRRKRRR [SEQ ID 5]; PHHP-6: ILVWWFWIWFWWASTRRKRRKRRR [SEQ ID 6].

3. The PHHP peptide of claim 2, wherein the PHHP peptide has the generic formula of (M)1-3 (N)4-6 (O)2-5 (P)7-10 where M is any one of the aromatic amino residues F, Y, or W or hydrophobic residues I, and/or L; N is any one or more of the following amino residues W, F, I, S, or L; O is one or more of G, A, P, T and S residues; and P is one or more charged positive amino residues R, and/or K and is interspersed with aromatic and hydrophobic residues F, W, Y, A, V.

4. The PHHP peptide according to claim 1, further comprising between 12 and 30 amino acid residues.

5. The PHHP peptide according to claim 4, wherein the peptide consists of between 21 and 24 amino acid residues.

6. The PHHP peptide according to claim 1, further comprising one or more of hydrophilic, hydrophobic, or amphipathic domain regions.

7. The PHHP peptide according to claim 1, further comprising a secondary structure having one or more of helix, beta strand, coil, turn, and loops.

8. The PHHP peptide according to claim 1, further comprising 1 to 3 repeating units of hydrophilic, hydrophobic, or amphipathic domain regions.

9. The PHHP peptide according to claim 6, wherein the domain regions have 3 to 30 amino acids.

10. The PHHP peptide according to claim 9, wherein the peptide is soluble in aqueous and organic solutions.

11. The PHHP peptide according to claim 10, wherein the peptide has a basic pH.

12. The PHHP peptide according to claim 11, wherein the peptide has an isolectric point greater than about 12.5.

13. The PHHP peptide according to claim 1, wherein the peptide has a positive charge at physiological pH.

14. Compositions comprising the peptide of claim 1.

15. Compositions comprising the peptide of claim 2.

16. The compositions of claim 15, further comprising polymeric, lipid, proteinaceous, carbohydrate, polysaccharide, or their combination particulate carriers.

17. The compositions of claim 15, further comprising a protein coding sequence joined with the peptide coding sequence and expressed in bacterial host cells.

18. The compositions of claim 15, further comprising fusion constructs of the peptide with therapeutic proteins.

19. Molecular assemblies, comprising the PHHP peptide of claim 1 complexed or conjugated with a nano- or micro-scale carrier.
